(19) United States
(12) Patent Application Publication
(10) Pub. No.: US 2017/0014534 A1
(43) Pub. Date: Jan. 19, 2017

(54) MALTOL-COATED MAGNETITE NANOPARTICLES, COMPOSITIONS AND METHODS COMPRISING SAME

(71) Applicant: INNOVATE CALGARY, Calgary (CA)

(72) Inventors: THOMAS W. CLEMENTS, Calgary (CA); CHRISTOPHER SARSONS, Calgary (CA); PATRICK LAI, Calgary (CA); ELMAR J. PRENNER, Calgary (CA); BARBARA BLASIAK, Calgary (CA); BOGUSLAW TOMANEK, Calgary (CA); KRISTINA D. RINKER, Calgary (CA); SIMON TRUDEL, Calgary (CA)

(21) Appl. No.: 15/157,941

(22) Filed: May 18, 2016

Related U.S. Application Data
(60) Provisional application No. 62/167,646, filed on May 28, 2015.

(51) Int. Cl. A61K 49/18

(52) U.S. Cl. CPC A61K 49/1833

(57) ABSTRACT

The present invention relates to maltol-coated magnetite nanoparticles, pharmaceutical compositions comprising same, and methods for using same as contrast agents in magnetic resonance imaging.

### FIELD OF THE INVENTION

The present invention relates to maltol-coated magnetite nanoparticles, pharmaceutical compositions comprising same, and methods for using same as contrast agents in magnetic resonance imaging.

### BACKGROUND OF THE INVENTION

Magnetic resonance imaging (MRI) is a powerful, high-resolution diagnostic tool for in vivo imaging of organs, soft tissues, bone, and other internal body structures without resorting to harmful ionizing radiation (x-rays). MRI operates on the basis of the response of hydrogen nuclei in water molecules to an applied static magnetic field. When placed in a sufficiently strong field, the hydrogen nuclei absorb energy from an applied intense electromagnetic radio frequency field which causes the hydrogen nuclei (“spins”) of water in tissues to be polarized in the direction of the magnetic field. The radio frequency field tips the magnetization generated by the hydrogen nuclei in the direction of the receiver coil where the spin polarization can be detected. Shortly thereafter the magnetization undergoes “relaxation” mechanisms that bring the net magnetization back to its equilibrium position in alignment with the applied magnetic field. Two relaxation mechanisms with characteristic relaxation times T₁ and T₂ are involved, called longitudinal (associated with T₁) and transverse relaxation (associated with T₁). The magnitude of the spin polarization detected by the receiver is used to form the MR and is dependent on both T₁ and T₂. Water protons in different tissues have different relaxation times, which is one of the main sources of contrast in MR images. A contrast agent usually shortens the value of T₁ and/or T₂ of nearby water protons, thereby altering the contrast in the image and assisting in clinical imaging.

However, due to the Boltzmann statistics of the energy levels probed during a magnetic resonance experiment, only about 0.001% of protons are detected (Lee et al., 2012). Furthermore both healthy and abnormal tissues have similar relaxation times, hindering MRI contrast. Indeed, most of MRI human scans require the use of contrast agents shortening relaxation times of abnormal tissues to improve contrast-to-noise ratio. Most clinical imaging is carried out at in magnetic resonance scanners operating with a field between 1.5 and 3 T. Imaging at ultra-high fields (>7 T) results in higher signal-to-noise ratios, thereby increasing spatial and temporal resolution (Blasiak et al., 2012). However relaxivity depends on the strength of the magnetic field, imposing specific contrasts depending on the field (Dong et al., 2012).

Magnetic nanoparticles have been intensively investigated due to their potential application as MRI contrast agents (Lee et al., 2012; Na et al., 2009; Blasiak et al., 2013). Iron oxide nanoparticles are of considerable interest due to their superparamagnetic nature since they strongly shorten T₂ relaxation time, have good water solubility, and acceptable biocompatibility (Lewinski et al., 2008).

For a nanoparticle with magnetic radius core a and impermeable coating thickness L, the spin-spin relaxation time R₂ is expressed as:

$${R}_{2} = \frac{1}{{T}_{2}} = \frac{256 {π}^{2} {γ}^{2}}{405} \frac{κ {m}_{s}^{2} {a}^{2}}{D ( 1 + \frac{L}{a} )} \qquad ( I )$$

wherein y is the gyromagnetic ratio of the proton, m₃ is the saturation magnetization of the nanoparticle sample, κ=V*/C_{Fe} where V is the volume fraction of the magnetic core, C_{Fe} is the concentration of Fe atoms, and D is the diffusion coefficient of water molecules (Lee et al., 2012; Tong et al., 2010). Inspection of equation (I) indicates avenues to improve R₂ by judiciously choosing the nanoparticles' surface coating. Water molecules must be close to the surface of the magnetic nanoparticle (obtained by decreasing the capping molecule's thickness L), and the residence time of water molecules in the vicinity of the magnetic core must be increased (by effectively decreasing the diffusion coefficient D).

A hydrophilic ligand able to hydrogen bond to solvent water molecules would have this desired effect. However, while poly(ethylene glycol) or dextran are well-known solubilizing ligands for metal-oxide nanoparticles, the long polymer length inevitably induces a larger hydrodynamic radius, maintaining water at a distance from the magnetic field produced by the nanoparticle's magnetic core (Tong et al., 2010).

Accordingly, there is a need in the art for improved contrast agents for use in MRI.

### SUMMARY OF THE INVENTION

The present invention relates to maltol-coated magnetite nanoparticles, pharmaceutical compositions comprising same, and methods for using same as contrast agents in magnetic resonance imaging.

In one aspect, the invention comprises a nanoparticle comprising a magnetite (Fe₃O₄) core, a non-magnetic layer surrounding the magnetite core, and a maltol coating formed on the non-magnetic layer, wherein the maltol coating comprises a maltol shell surrounding the non-magnetic layer and the magnetite core.

In one aspect, the invention comprises a pharmaceutical composition comprising the above nanoparticle in combination with one or more pharmaceutically acceptable carriers.

In another aspect, the invention comprises a magnetic resonance imaging contrast agent for detecting an in vivo site by magnetic resonance imaging, comprising the above nanoparticle.

In yet another aspect, the invention comprises a method of in vivo imaging of a site within a subject comprising administering to the subject a nanoparticle, or a pharmaceutical composition comprising the nanoparticle in combination with one or more pharmaceutically acceptable carriers; and imaging the site of the subject.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a schematic representation of one embodiment of a method for producing the maltol-coated magnetite nanoparticles of the present invention.

FIG. 2 shows UV-vis spectra of uncoated (dotted lines) and maltol-coated (solid lines) magnetite nanoparticles in water (room temperature, 100 μg mL⁻¹).

FIG. 3A is a transmission electron microscopy (TEM) image of maltol-coated magnetite nanoparticles.

FIG. 3B shows the associated size distribution (percent frequency plotted against diameter in nm) of the nanoparticles of FIG. 3A.

FIG. 3C is a field cooled/zero field cooled (FC/ZFC) magnetization curve showing the magnetization M (expressed as emu g⁻¹) of the nanoparticles of FIG. 3A plotted against temperature (Kelvin) indicating a blocking temperature (T_{B}) of about 190 K.

FIG. 3D is a magnetization vs field curve showing the magnetization M (expressed as emu g⁻¹) of the nanoparticles of FIG. 3A plotted against permeability of free space (μ₀)×magnetic field intensity (H (expressed as Teslas T) indicating superparamagnetic behavior at 300 K with mₛ=26.8 emu/g.

FIG. 4 shows a powder X-ray diffractogram of maltol-coated magnetite nanoparticles synthesized at 70° C., with all observed peaks corresponding to magnetite (Fe₃O₄) (JCPDS Card No. 75-0033).

FIG. 5 shows FTIR spectra (KBr disks) of maltol, maltol-coated magnetite nanoparticles and uncoated (“bare”) magnetite nanoparticles.

FIG. 6 is a schematic representation explaining variables in equation (III), where the nanoparticle's magnetite inner core (i.e. the TEM-determined size) is r_{core} and d is the thickness of a low-magnetization or non-magnetic magnetite layer.

FIGS. 7A-B show graphs of measured longitudinal (T₁) and transverse (T₂) relaxation times (B₀=9.4 T) (FIG. 7A) and associated relaxation rates Rᵢ=Tᵢ⁻¹ (FIG. 7B). Lines are fits to T₁⁻¹=T_{0.1}⁻¹+r₁C with relaxivities r₁=2.11±0.02 mM⁻¹ s⁻¹ and r₂=191±15 mM⁻¹ s⁻¹.

FIGS. 8A-B show graphs of measured longitudinal (T₁) and transverse (T₂) relaxation times (B₀=3.0 T) (FIG. 8A) and associated relaxation rates Rᵢ=Tᵢ⁻¹ (FIG. 8B). Lines are fits to Tᵢ⁻¹=T_{0,i}⁻¹+rᵢC with relaxivities r₁=1.03±0.05 mM⁻¹ s⁻¹ and r₂=126±12 mM⁻¹ s⁻¹.

FIG. 9A shows confocal microscope images of HUVECs exposed to maltol-coated magnetite nanoparticles for 24 h in a membrane permeability cytotoxicity assay.

FIG. 9B shows quantification of cell viability of HUVEC, KB and U343 cells.

FIG. 10 shows confocal microscope images of HUVECs exposed to the maltol-coated magnetite nanoparticles for 24 hours at a range of exposure concentrations.

FIG. 11 shows confocal microscope images of KB cells exposed to the maltol-coated magnetite nanoparticles for 24 hours at a range of exposure concentrations.

FIG. 12 shows confocal microscope images of U343 cells exposed to the maltol-coated magnetite nanoparticles for 24 hours at a range of exposure concentrations.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a”, “an”, and “the” include plural referents unless the context clearly dictates otherwise.

The present invention relates to a magnetite nanoparticle which is coated entirely or partially with maltol. As used herein, the term “magnetite” refers to an iron oxide having the chemical formula Fe₃O₄. As used herein, the term “nanoparticle” means a particle having at least one dimension which is less than about 200 nm.

As used herein, the term “maltol” refers to a naturally occurring organic compound which is commonly used as a flavor enhancer, and may be sourced from the bark of larch trees, pine needles, and roasted malt. The chemical name is 3-hydroxy-2-methyl-4H-pyran-4-one. Synonyms and other names for “maltol” include larixinic acid, palatone, and veltol. Maltol is a white crystalline powder which is soluble in hot water, chloroform, and other polar solvents, and readily chelates to metal ions including Fe³⁺, Ga³⁺, Al³⁺, and VO²⁺ (Thompson et al., 2006; Ahmet et al., 1988). The empirical formula of maltol is C₆H₆O₃. The structural formula is:

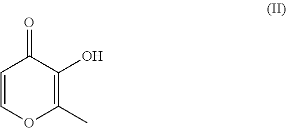

Compounds related to maltol or derivatives of maltol include, but are not limited to, other 3-hydroxy-4-pyrones such as, for example, 5-hydroxy-2-(hydroxymethyl)-4H-pyran-4-one or kojic acid; 2-ethyl-3-hydroxy-4-pyranone or ethyl maltol; Tris(3-hydroxy-2-methyl-4H-pyran-4-one) gallium or gallium maltolate; 3,5-dihydroxy-2-methylpyran-4-one or 5-hydroxy maltol; and 1-(3-hydroxy-2-furanyl)ethanone or isomaltol.

There is very minimal systemic toxicity associated with using maltol and its related or derived compounds for medical applications. The nanoparticles of the present invention are thus biodegradable and biocompatible, and have a magnetite core making them superparamagnetic. In addition, maltol acts as a capping ligand which provides good water solubility, thereby imparting enhanced medical imaging capabilities. The nanoparticles can serve as suitable contrast agents for medical imaging, particularly magnetic resonance imaging (MRI). As used herein, the term “contrast agent” means a substance used to improve the visibility of internal body structures by enhancing the contrast of such structures in medical imaging. As described herein, the maltol-coated magnetite nanoparticles achieve high T₂ contrast performance in ultrahigh magnetic field.

In one embodiment, the nanoparticle comprises a magnetite (Fe₃O₄) core, a non-magnetic layer surrounding the magnetite core, and a maltol coating formed on the non-magnetic layer, wherein the maltol coating comprises a maltol shell surrounding the non-magnetic layer and the magnetite core.

In one embodiment, the non-magnetic layer comprises magnetically disordered magnetite (Fe₃O₄). In one embodiment, the non-magnetic layer has a thickness of about 3.5 nm. The maltol shell can entirely or partially surround the non-magnetic layer and the magnetite core. In one embodiment, the maltol shell entirely surrounds the non-magnetic layer and the magnetite core. In one embodiment, the maltol shell comprises at least one maltol moiety bound to the non-magnetic layer. In one embodiment, the maltol moiety is chelated to the non-magnetic layer.

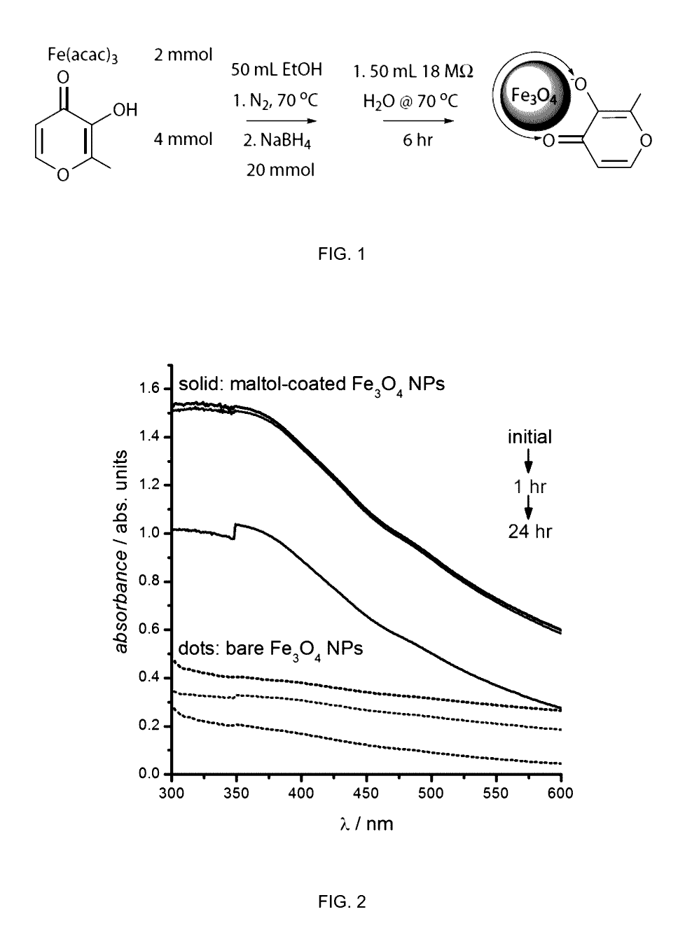

Exemplary nanoparticles are prepared using a modified reductive one-pot synthesis as shown in FIG. 1 (Yathindranath et al., 2011; 2013) and described in Example 2. Briefly, absolute ethanol and water are each deoxygenated by bubbling nitrogen gas through each liquid. In one embodiment, deoxygenation is conducted for at least about thirty minutes. In one embodiment, the water is ultra purified water. Both liquids are then heated. In one embodiment, the temperature is about 70° C. Tris(acetylacetonato) iron(III) and maltol are dissolved in the degassed ethanol, and sodium borohydride is rapidly added to the mixture. The degassed water is then added. In one embodiment, the degassed water is added about ten minutes after the addition of sodium borohydride. The resultant mixture is stirred. In one embodiment, the mixture is stirred for about six hours. The resultant maltol-coated magnetite nanoparticles are separated magnetically, washed twice with absolute ethanol, and centrifuged twice with absolute ethanol. In one embodiment, centrifugation is conducted at about 6000 RPM for about 10 minutes. The nanoparticles are dried in air overnight.

Exemplary nanoparticles were confirmed to be water soluble, as determined by ultraviolet-visible spectroscopy (FIG. 2). The O-atom in the ring of maltol confers water solubility to the magnetite nanoparticle. Maltol-coated magnetite nanoparticles are more soluble in water than uncoated magnetite nanoparticles, exhibit little precipitation within one hour, and still have a fair concentration when left undisturbed for 24 hrs. The results indicate that maltol-coated magnetite nanoparticles or pharmaceutical compositions comprising same may thus remain stable for an extended time period such as, for example, days or months.

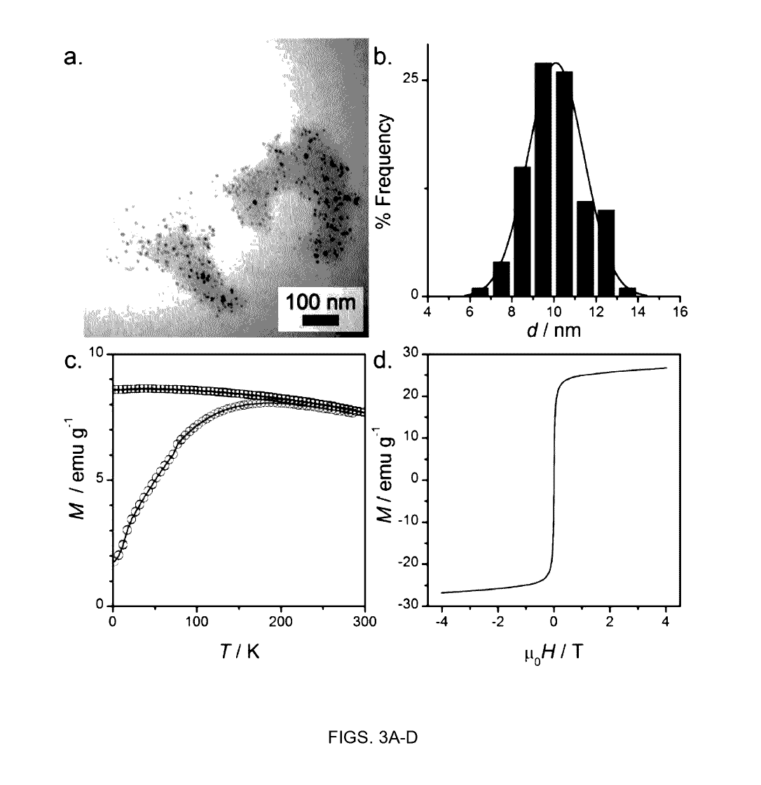

The nanoparticles were confirmed to be spherical, as observed by transmission electron microscopy (FIG. 3A). The superparamagnetic properties of nanoparticles are dependent on particle size (FIG. 3B). In general, nanoparticles below 20 nm retain superparamagnetism and influence the T₂ relaxation (Yathindranath et al., 2011; Cornell et al., 2004). In one embodiment, the nanoparticle has a diameter ranging from about 5 nm to about 15 nm. In one embodiment, the nanoparticle has an average diameter of about 10 nm.

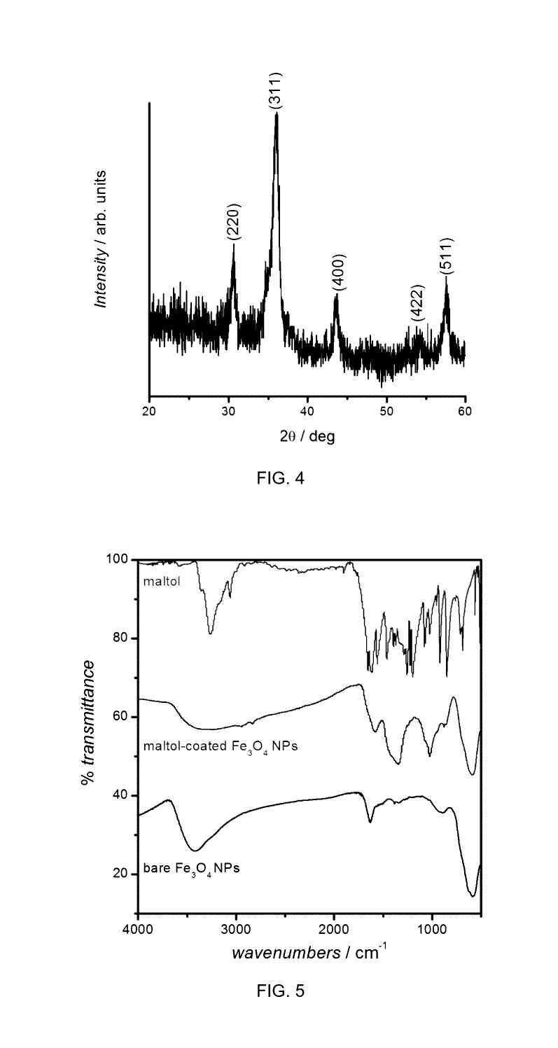

The nanoparticles comprise magnetic cores of magnetite, as confirmed by powder X-ray diffraction (FIG. 4) and Fourier-transform infrared (FTIR) spectroscopy (FIG. 5), The strong band in the 500-750 cm⁻¹ is characteristic of various iron oxides, in particular the closely related magnetite and maghemite (Cornell et al., 2004). The IR absorption band at 590 cm⁻¹ is assigned to the ν (Feoct-O-Feoct) stretch in iron oxide.

The binding of maltol to the surfaces of the magnetite nanoparticles was confirmed using FTIR spectroscopy (FIG. 5). Maltol is bound to the surfaces of the magnetite nanoparticles through chelation. As used herein, the term “chelation” refers to the formation or presence of two or more separate coordinate bonds between a polydentate (multiple bonded) ligand and a single central atom. The bands at 1590, 1360, and 1030 are assigned to ν(C═O), δₐₛ(CH₃), and νₐₛ(C—O—C)+ν_{ring} vibrations of the maltotato ligand, respectively (Parajón-Costa et al., 2013). The band at 590 cm⁻¹ is assigned to the ν(Fe_{oct}—O—Fe_{td}) in Fe₃O₄ (Cornell et al., 2003).

The nanoparticles of the present invention are superparamagnetic. As used herein, the term “superparamagnetic” means that the nanoparticle sample as a whole is not magnetized unless it is subjected to an external magnetic field, and can be detected during, for example, magnetic resonance imaging and the like. In sufficiently small nanoparticles, magnetization can randomly flip direction under the influence of temperature. The typical time between two flips is called the Néel relaxation time. In the absence of an external magnetic field, when the time used to measure the magnetization of the nanoparticles is much longer than the Néel relaxation time, their magnetization appears to be in average zero (“superparamagnetic state”). In this state, an external magnetic field is able to magnetize the nanoparticles. Superparamagnetism may be determined using a superconducting quantum interference device magnetometer.

In one embodiment, the nanoparticles exhibit a blocking temperature T_{B} of about 190 K (FIG. 3C). In one embodiment, the nanoparticles exhibit a saturation magnetization of about 26.8 emu g⁻¹ at 300 K under a 4 T field (FIG. 3D). This low saturation magnetization value (compare to the saturation magnetization of bulk iron oxide of about 92 emu g⁻¹) is consistent with the presence of a magnetically disordered (i.e., non-magnetic) magnetite surface layer of thickness d. This reduced magnetization is often observed in ferrite nanoparticles (Na et al., 2009; Jun et al., 2008). Saturation magnetization is expressed as:

$${m}_{s} = {{M}_{s} ( \frac{{r}_{core} - d}{{r}_{core}} )}^{3} \qquad ( III )$$

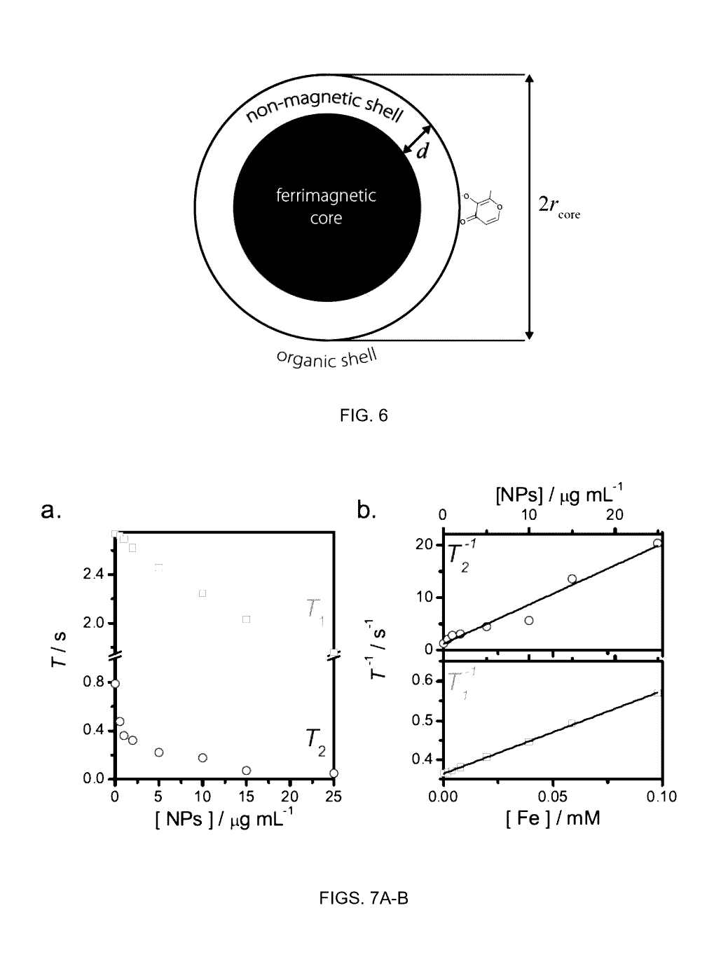

The obtained saturation magnetization is consistent with a non-magnetic surface layer of thickness d of about 3.4 nm, where r_{core} is the radius of the magnetite core (FIG. 6). Without being bound by any theory, this is presumably due to the low temperature used during the synthesis which leads to poor crystallinity and lowered magnetization. Higher reaction temperatures may lead to more magnetic nanoparticles, a factor expected to improve the MRI capabilities of these materials.

The magnetic resonance properties of the maltol-coated magnetite nanoparticles are evaluated using an ultra-high-field MRI scanner operating for example at about 9.4 T using the protocol described in Das et al. (2012). The longitudinal (T₁) and transverse (T₂) relaxation times (s) are measured as a function of nanoparticle concentration (μg/mL) to determine the respective relaxivities (r₁ and r₂) (FIG. 7A). In one embodiment, r₁ ranges from about 2.09 to about 2.13 mM⁻¹ s⁻¹. In one embodiment, r₂ ranges from about 176 to about 206 mM⁻¹ s⁻¹ (FIG. 7B).

The r₂ relaxivity of the maltol-coated magnetite nanoparticles measured at about 9.4 T compares favorably with those of commercial iron-oxide-nanoparticle-based contrast agents (Table 1; Na et al., 2009). The r₂ is on par (Resovist, r₂=186 mM⁻¹ s⁻¹) or exceeds (Feridex r₂=120 mM⁻¹ s⁻¹, Combidex r₂=65 mM⁻¹ s⁻¹) those found for these materials, despite the reduced saturation magnetization (compare to about 45-60 emu g⁻¹ for these agents).

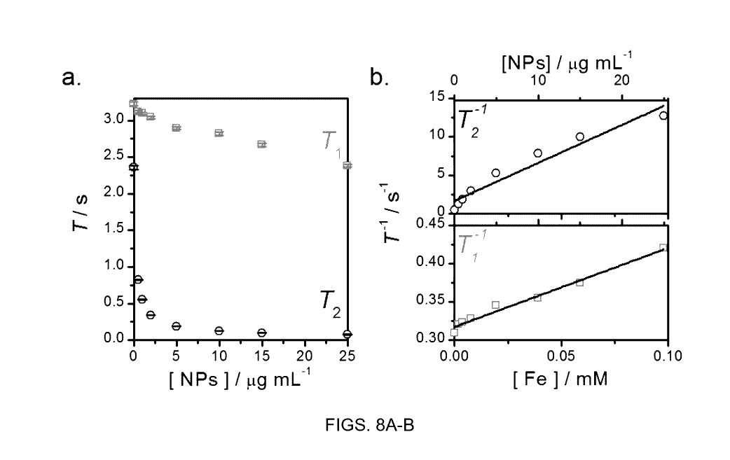

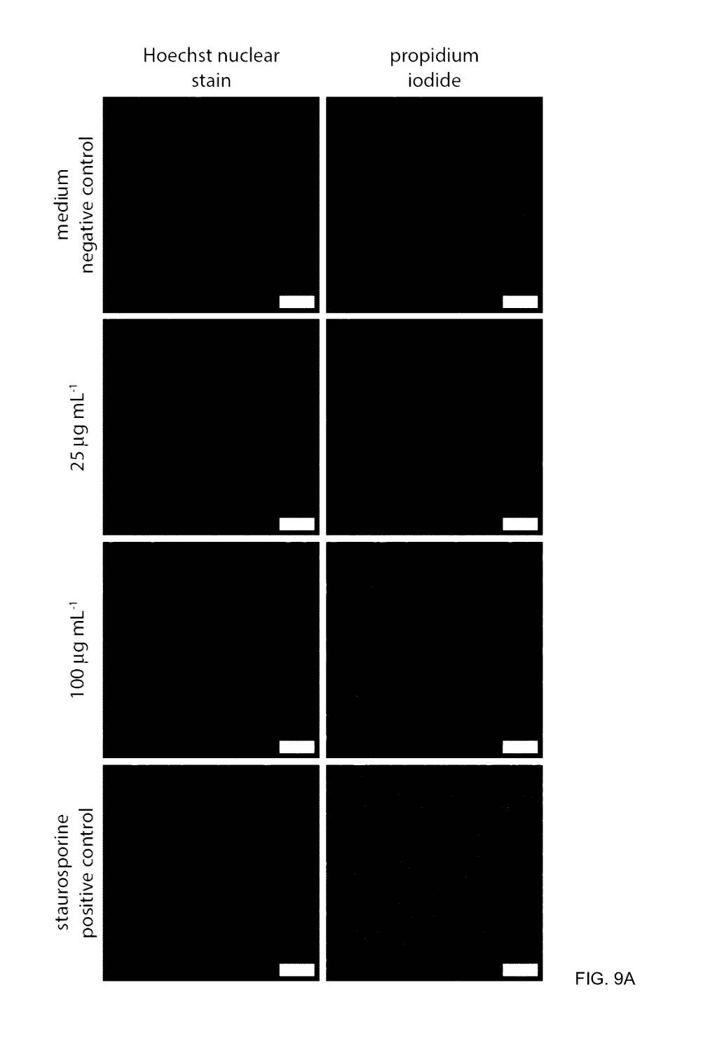

The magnetic resonance properties of the maltol-coated magnetite nanoparticles are evaluated using a clinical MRI scanner operating for example at about 3.0 T. The longitudinal (T₁) and transverse (T₂) relaxation times (s) are measured as a function of nanoparticle concentration (μg/mL) to determine the respective relaxivities (r₁ and r₂) (FIG. 8A). In one embodiment, r₁ ranges from about 0.98 to about 1.08 mM⁻¹ s⁻¹. In one embodiment, r₂ ranges from about 114 to about 138 mM⁻¹ s⁻¹ (FIG. 8B).

The r₂ relaxivity of the maltol-coated magnetite nanoparticles measured at about 3.0 T compares favorably with those of commercial iron-oxide-nanoparticle-based contrast agents (Table 1; Na et al., 2009). The r₂ is on par (Feridex r₂=120 mM⁻¹ s⁻¹) or exceeds (Combidex r₂=65 mM⁻¹ s⁻¹) those found for these materials, despite the reduced saturation magnetization (compare to about 45-60 emu g⁻¹ for these agents).

TABLE 1
Transverse relaxivities r₂ for various benchmark
iron-oxide nanoparticle contrast agents

| Sample | r₂/mM⁻¹s⁻¹ | B₀/T | Reference |
|---|---|---|---|
| Octapod FeOₓ | 679 ± 30 | 7 | Zhao et al., 2013 |
| FION + | 324 | 1.5 | Lee et al., 2011 |
| PEG-phospholipid | | | |
| WSIO + | 218 | 1.5 | Jin et al., 2005 |
| 2,3-dimercaposuccinic acid | | | |
| Fe₃O₄ + maltol | 191 ± 15 | 9.4 | Present |
| | | | application |
| | 126 ± 12 | 3.0 | Present |
| | | | application |
| Fe₃O₄ + carboxydextran | 186 | 1.5 | Jung et al., 1995 |
| (Resovist ™) | | | |
| Fe₃O₄ γ-Fe₂O₃ + dextran | 120 | 1.5 | Wang et al., 2001 |
| (Feridex ™) | | | |
| Fe₃O₄ + dextran | 65 | 1.5 | Wang et al., 2001 |
| (Combidex ™) | | | |

A figure of merit for the efficacy of an MRI contrast agent is the r₂/r₁ ratio (Terreno et al., 2010; Qin et al., 2007). The maltol-coated magnetite nanoparticles exhibit a high r₂/r₁ of 90.5 at 9.4 T, demonstrating their potential for ultra-high field imaging applications. This ratio is three-fold that of commercial iron-oxide nanoparticle agents at clinical fields (≦3 T) as shown in Table 2.

A figure of merit for the efficacy of an MRI contrast agent is the r₂/r₁ ratio (Terreno et al., 2010; Qin et al., 2007). The maltol-coated magnetite nanoparticles exhibit a high r₂/r₁ of 126 at 3.0 T, demonstrating their potential for ultra-high field imaging applications. This ratio is up to 20-fold that of commercial iron-oxide nanoparticle agents at clinical fields (≦3 T) as shown in Table 2.

TABLE 2
Ratio of the transverse-to-longitudinal relaxitivities (r₂/r₁)
for various benchmark iron-oxide nanoparticle contrast agents

| Sample | r₂/r₁ | B₀/T | Reference |
|---|---|---|---|
| Fe₃O₄ + maltol | 91 ± 7 | 9.4 | Present application |
| | 122 ± 13 | 3.0 | Present application |
| Fe₃O₄ + carboxydextran | 31 | 3 | Das et al., 2012 |
| (Resovist ™) | | | |
| Fe₃O₄ γ-Fe₂O₃ + dextran | 22 | 3 | Das et al., 2012 |
| (Feridex ™) | | | |
| | 10.1 | 1.4 | Lee et al., 2012 |
| Fe₃O₄ + dextran (Combidex ™) | 6 | 1.5 | Das et al., 2001 |
| Fe₃O₄ + dextran | 2.11 | 0.47 | Lee et al., 2012 |
| γ-Fe₂O₃ + PEG | 4.77 | 3 | Lee et al., 2012 |
| Fe/MnFe₂O₄ core/shell + | 32 | 0.47 | Lee et al., 2012 |
| 2,3-dimercaposuccinic acid | | | |

The nanoparticles are non-toxic and clinically applicable, as evaluated in vitro in human cell types using a propidium iodide (PI) based assay. Variability in cytotoxic response between cells of different origins and between primary cells and immortalized cell lines has been observed, imposing the need for experimentation using multiple cell types (Hu et al., 2010; Hanley et al., 2008). Two immortalized cell lines from oral epithelial (KB) and neuronal (U343) and one type of primary endothelial cells (human umbilical vein endothelial cells, HUVEC) were thus selected.

The cells were exposed to the maltol-coated magnetite nanoparticles in concentrations ranging from 2.5 to 250 μg mL⁻¹ for 24 hours. The range in exposure concentrations covers two orders of magnitude and brackets the expected physiological exposure concentrations of the nanoparticles based on similar particles, 6 to 140 μg mL⁻¹ (Wang et al., 2001). The upper limit of 250 μg mL⁻¹ also corresponds roughly with the solubility limit of the maltol-coated magnetite nanoparticles in culture media. The exposure time of 24 hours is consistent with many in vitro nanocytotoxicity protocols, facilitating comparison (Jan et al., 2008; Gupta et al., 2004; Lin et al., 2006; Thomassen et al., 2010). Additionally, the clearance half-life of iron oxide based contrast agents ranges from several minutes to 36 hours, 26 indicating that 24 hours is a well-placed time point to observe acute cytotoxicity.

Following exposure, the cells were treated with PI, a fluorescent molecule that selectively permeates the membrane of dead cells. The more permeable membranes of dead cells are successfully penetrated by the dye, providing a fluorescence signal whose absence is indicative of cell viability, following the removal of any remaining extracellular dye. The cells were counterstained with Hoechst cell nucleus dye and imaged using a laser scanning confocal microscope. Cell viability was calculated by subtracting the number of PI positive cells from the total cell counts.

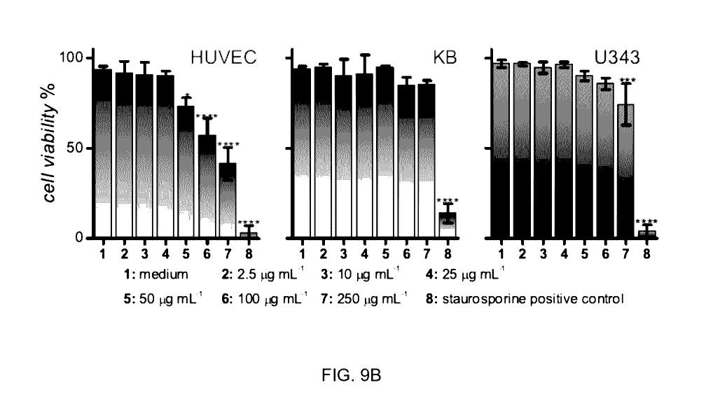

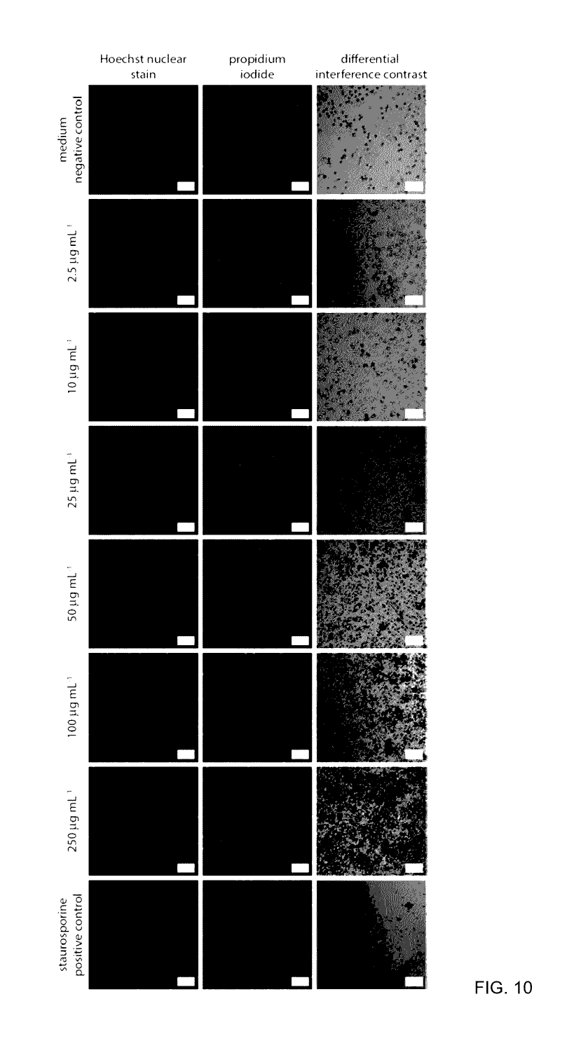

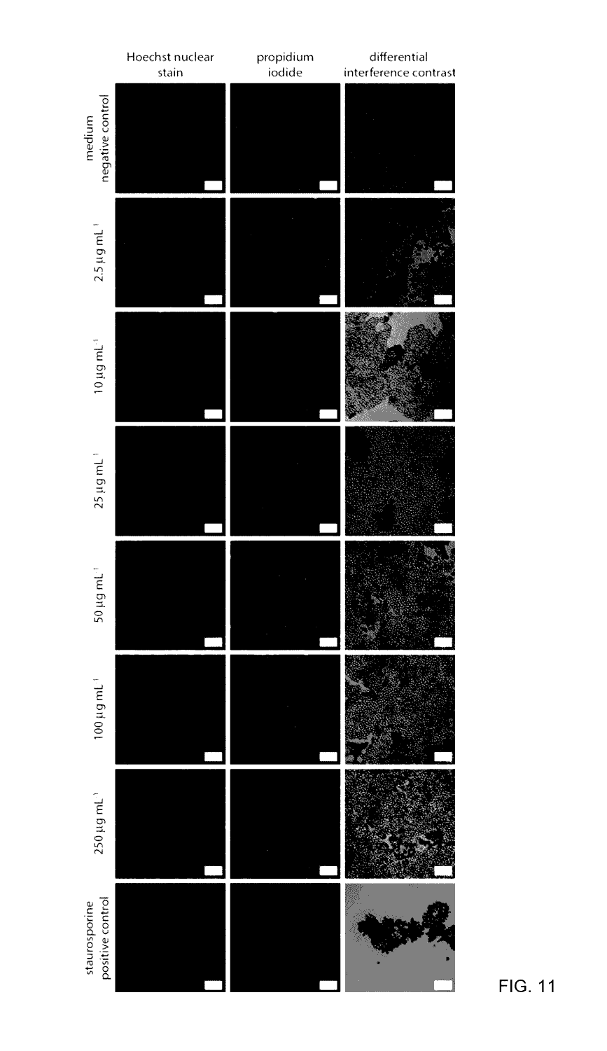

The viability of the cells was imaged and quantified using confocal microscopy. FIG. 9A shows confocal microscope images of HUVECs exposed to maltol-coated magnetite nanoparticles for 24 h in the membrane permeability cytotoxicity assay. Cell nuclei stained with Hoechst provided a cell count, while dead cells were stained with PI (scale bars: 100 Lm). FIG. 9B shows quantification of cell viability of HUVEC, KB and U343 cells (error bars=standard deviation of N=3 replicate samples; *p<0.05, <0.001, ****p<0.0001). The HUVECs displayed the greatest cytotoxic response to the nanoparticles, with statistically significant toxicity at concentrations of 50 μg mL⁻¹ and greater. The mean viability of the HUVEC cells decreased from 94% for the negative control, to 73% at 50 μg/mL exposure and finally to 41% viability at an exposure concentration of 250 μg mL⁻¹. The KB cells did not have any cytotoxic response to the nanoparticles in the tested concentration range, and the U343 cells only demonstrated toxicity at the 250 μg mL⁻¹ exposure concentration with a mean viability of 74% (compared to a negative control viability of 97%).

FIG. 10 shows confocal microscope images of HUVECs exposed to the maltol-coated magnetite nanoparticles for 24 hours at a range of exposure concentrations. The blue fluorescence channel shows the cell nuclei stained with Hoechst, the red fluorescence channel shows the dead cells stained with propidium iodide and the third channel contains differential interference contrast (DIC) images. A 10× magnification, 0.3 numerical aperture, water immersion objective was used for all images with 2× digital zoom. The scale bar represents 100 μm. The growth medium was EGM-2.

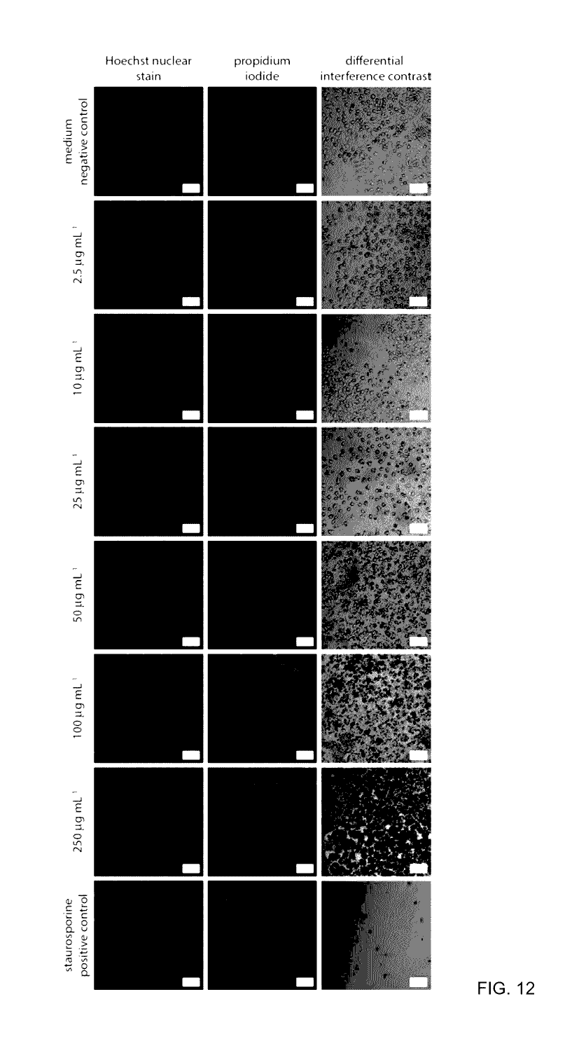

FIG. 11 shows confocal microscope images of KB cells exposed to the maltol-coated magnetite nanoparticles for 24 hours at a range of exposure concentrations. The blue fluorescence channel shows the cell nuclei stained with Hoechst, the red fluorescence channel shows the dead cells stained with propidium iodide and the third channel contains differential interference contrast (DIC) images. A 10× magnification, 0.3 numerical aperture, water immersion objective was used for all images with 2×digital zoom. The scale bar represents 100 m. The growth medium was MEM.

FIG. 12 shows confocal microscope images of U343 cells exposed to the maltol-coated magnetite nanoparticles for 24 hours at a range of exposure concentrations. The blue fluorescence channel shows the cell nuclei stained with Hoechst, the red fluorescence channel shows the dead cells stained with propidium iodide and the third channel contains differential interference contrast (DIC) images. A 10× magnification, 0.3 numerical aperture, water immersion objective was used for all images with 2× digital zoom. The scale bar represents 100 μm. The growth medium was DMEM.

All three cell types are non-toxic for the majority, or all, of the expected physiological concentration ranges, and are generally comparable to various other investigated iron-oxide nanoparticles (Lewinski, 2008). It seems plausible that the toxicity of the nanoparticles at the 250 μg mL⁻¹ exposure concentration is exaggerated due to the limited solubility of the nanoparticles at this concentration in culture media. A blanketing of nanoparticles on top of the cells is visible in the differential interference contrast images at this concentration (FIGS. 10-12), indicating that the dispersion may not have been stable over the 24 hours of exposure. Significant nanoparticle settling over the exposure time would increase the effective exposure concentration, exaggerating any toxic effects.

The nanoparticles of the present invention may be formulated for use in medical imaging. In one embodiment, the invention comprises a pharmaceutical composition comprising the nanoparticle in combination with one or more pharmaceutically acceptable carriers. As used herein, the term “carrier” means a suitable vehicle which is biocompatible and pharmaceutically acceptable, including for instance, liquid diluents which are suitable for administration. As used herein, the term “biocompatible” means generating no significant undesirable host response for the intended utility. Most preferably, biocompatible materials are non-toxic for the intended utility. Thus, for human utility, biocompatible is most preferably non-toxic and otherwise non-damaging to humans or human tissues. As used herein, the term “pharmaceutically acceptable” means a substance which does not significantly interfere with the effectiveness of the compound, and which has an acceptable toxic profile for the host to which it is administered.

Suitably, pharmaceutical compositions comprising the nanoparticles may in various embodiments be formulated for administration orally, intravenously, rectally, intravascularly, or by injection or infusion techniques in dosage unit formulations containing conventional non-toxic pharmaceutically acceptable carriers, adjuvants and vehicles.

Compositions intended for oral use may be prepared in either fluid or solid unit dosage forms. Contrast agents are typically prepared in fluid unit dosage forms which the subject can readily ingest prior to the MRI. Fluid unit dosage form can be prepared according to procedures known in the art for the manufacture of pharmaceutical compositions and such compositions may contain one or more agents such as sweetening agents, flavouring agents, colouring agents and/or preserving agents in order to provide pharmaceutically elegant and palatable preparations.

Contrast agent medication may be taken an evening before the MRI test, and may be prepared in solid unit dosage forms. Solid formulations such as tablets contain nanoparticles in admixture with non-toxic pharmaceutically acceptable excipients that are suitable for the manufacture of tablets or other solid formulations. These excipients may be for example, inert diluents, such as calcium carbonate, sodium carbonate, lactose, calcium phosphate or sodium phosphate; granulating and disintegrating agents for example, corn starch, or alginic acid; binding agents, for example starch, gelatin or acacia; and lubricating agents, for example magnesium stearate, stearic acid or talc and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over a longer period.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the compound with an acceptable vegetable oil, light liquid petrolatum or other inert oil.

Aqueous suspensions may contain nanoparticles in admixture with excipients suitable for the manufacture of aqueous suspensions. Such excipients are suspending agents, for example sodium carboxylmethylcellulose, methyl cellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or w-propyl-p-hydroxy benzoate, one or more colouring agents, one or more flavouring agents or one or more sweetening agents, such as sucrose or saccharin.

Oily suspensions may be formulated by suspending the nanoparticles in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the nanoparticles in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions may also be in the form of oil-in-water emulsions. The oil phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

The pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent. Among the acceptable vehicles and solvents that may be employed are water, Ringer's solution and isotonic sodium chloride solution. In addition, sterile, fixed oils may be employed as a solvent or suspending medium. Adjuvants such as preservatives and buffering agents may optionally also be included in the injectable solution or suspension.

The dosage of the pharmaceutical composition depends upon many factors that are well known to those skilled in the art, for example, the type and pharmacodynamic characteristics of the pharmaceutical composition; age, weight and general health condition of the subject; the internal site which is to be imaged; the nature and extent of symptoms; and the imaging method.

The pharmaceutical compositions comprising nanoparticles of the present invention will be useful as contrast agents in any MRI method or system known to those skilled in the art where it is desired to obtain an image of an internal or in vivo site. In one embodiment, the invention comprises a method of in vivo MRI imaging of a site within a subject comprising administering to the subject a nanoparticle, or a pharmaceutical composition comprising the nanoparticle in combination with one or more pharmaceutically acceptable carriers; and imaging the site of the subject. As used herein, the term “subject” means humans or other vertebrates. As used herein, the term “site” means any organ, soft tissue, bone, and other internal body structure. The term includes organs of the chest and abdomen including the heart, liver, biliary tract, kidneys, spleen, bowel, pancreas, and adrenal glands; pelvic organs including the bladder and the reproductive organs such as the uterus and ovaries in females and the prostate gland in males; blood vessels; and lymph nodes. The site is imaged to diagnose or monitor treatment for conditions including, but not limited to, tumors of the chest, abdomen or pelvis; diseases of the liver such as cirrhosis, and abnormalities of the bile ducts and pancreas; inflammatory bowel disease such as Crohn's disease and ulcerative colitis; heart problems such as congenital heart disease; malformations of the blood vessels and inflammation of the vessels; and a fetus during pregnancy. As used herein, the term “administering” means any action that results in exposing or contacting nanoparticles or a pharmaceutical composition comprising the nanoparticles of the present invention with any pre-determined internal site within the subject's body.

Embodiments of the present invention are described in the following Examples, which are set forth to aid in the understanding of the invention, and should not be construed to limit in any way the scope of the invention as defined in the claims which follow thereafter.

### Example 1

### Chemicals

Iron(III) 2,4-pentadione [Fe(acac)₃, 99%] was obtained from Strem, while maltol (3-hydroxy-2-methyl-4H-pyran-4-one, ≧99%) and sodium borohydride (NaBH₄, ≧96%) were obtained from Sigma-Aldrich. All chemicals and solvents were used as-received without further purification.

### Example 2

### Synthesis of Iron Oxide (Fe₃O₄) Nanoparticles

All nanoparticles were synthesized by adapting a reductive one-pot synthesis (Yathindranath et al., 2011). All synthetic steps were conducted under an inert N₂ atmosphere. Separation, washing and centrifuging were conducted under normal atmospheric conditions.

a) Uncoated Iron Oxide Nanoparticles

Absolute ethanol (50 mL) and Milli-Q™ water (50 mL) were deoxygenated by bubbling N₂ gas through the liquid for 30 minutes. Both liquids were then heated to 70° C. Fe(acac)₃ (0.7063 g, 2 mmol) was dissolved in the ethanol. NaBH₄ (0.7560 g, 20 mmol) was then added to the solution and gas was furiously evolved. Upon addition of NaBH₄, the solution color changed from red to brown to black. After 10 minutes, the 50 mL of degassed H₂O were added, and the resultant solution was magnetically stirred under N₂ at 70° C. for 6 hours. After 6 hours, the nanoparticles were separated magnetically, washed twice with absolute ethanol, and centrifuged twice with ethanol (6000 rpm, 10 minutes). The particles were left to dry overnight in air and collected.

b) Maltol-Coated Iron Oxide (Fe₃O₄) Nanoparticles

Absolute ethanol (50 mL) and 18 MΩ H₂O (50 mL) were deoxygenated by bubbling N₂ gas through the liquid for 30 minutes. Both liquids were then heated to 70° C. Fe(acac)₃ (0.7063 g, 2 mmol) was dissolved in the ethanol, along with maltol (0.5049 g, 4 mmol). NaBH₄ (0.7571 g, 20 mmol) was then added to the solution and gas was furiously evolved. Upon addition of NaBH₄, the solution color changed from red to brown to black. After 10 minutes, the 50 mL of degassed H₂O were added, and the resultant solution was magnetically stirred under N₂ at 70° C. for 6 hours. After 6 hours, the nanoparticles were separated magnetically, washed twice with absolute ethanol, and centrifuged twice with ethanol (6000 RPM, 10 minutes). The particles were left to dry overnight in air and collected.

### Example 3

### Characterization of the Maltol-coated Iron Oxide Nanoparticles

a) Transmission Electron Microscopy

The nanocrystals were imaged in bright field mode using a FEI Tecnai 20 transmission electron microscope, operating at 200 kV. Maltol-coated nanoparticles were dissolved in water, and a drop of the solution was allowed to evaporate on a carbon-coated copper grid.

b) Fourier-Transform Infrared Spectroscopy

Infrared spectra of nanoparticles in a KBr disk were collected on a Nicolet Nexus 470 spectrometer.

c) Thermogravimetric Analysis

Thermogravimetric analysis was recorded on a Netzsch STA 409 PC/PG apparatus under nitrogen at a heating rate of 2° C./min.

d) Powder X-Ray Diffraction

X-ray diffraction patterns were obtained using a Rigaku Multiflex (0-20) diffractometer (scan speed=0.016° min⁻¹, Cu Kα tube, λ=1.5406 Å).

e) Superconducting Quantum Interference Device (SQUID) Magnetometry

All measurements were carried out using a Quantum Design MPMS XL-7S SQUID magnetometer. Samples were loaded into gelatin capsules sealed with Kapton tape, which were themselves inserted in clear diamagnetic straws. Zero-field cooled (ZFC) and field cooled (FC) measurements were carried out by cooling the samples from 300 to 1.8 K in the absence (ZFC) or presence (FC, poH=10 mT) of an applied magnetic field. A field of 10 mT was then applied, and the magnetization of the sample measured upon warming from 1.8 to 300 K. Isothermal magnetization as a function of applied magnetic field strength measurements were also carried out at 300 and 5 K.

f) Ultraviolet-Visible (UV-Vis) Spectroscopy

UV-vis spectra of nanoparticles in water (room temperature, 100 μg mL⁻¹) were collected on a Cary-5E UV-vis-NIR spectrometer.

g) Relaxitivity at 9.4 T

The MRI experiments are detailed in Das et al. (2012). Briefly, T₁ and T₂ relaxation measurements and phantom images were obtained using a 9.4 T/21 cm magnet (Magnex, UK) and a Bruker console (Bruker, Germany). Standard 5-mm diameter NMR tubes were used for experiments. A transmit/receive radio frequency (RF) volume birdcage coil was applied. A single slice multi-echo pulse sequence was used for T₂ measurements with the following parameters: repetition time (T_{R})=7.5 s, 1 average, matrix size 128×128, field of view (FOV) 3 cm×3 cm, slice thickness 2 mm, 64 echoes 4 ms apart. The T₂ relaxation time was calculated using a single exponential fitting of the echo train (Bruker, Germany). For T₁ measurements TRUE FISP method was used with the following parameters: slice thickness 2 mm, FOV 3×3 cm, 1 average, matrix size 128×128, echo time (T_{E})=1.5 ms, T_{R}=1 s.

h) Relaxitivity at 3.0 T

Briefly, T₁ and T₂ relaxation measurements and phantom images were obtained using a 3.0 T system (Siemens, Germany). Standard 5-mm diameter NMR tubes were used for experiments. A transmit/receive radio frequency (RF) volume birdcage coil was applied. A single slice multi-echo pulse sequence was used for T₂ measurements with the following parameters: repetition time (T_{R})=15 s, 1 average, matrix size 128×128, field of view (FOV) 16 cm×16 cm, slice thickness 5 mm, 64 echoes 4 ms apart. The T₂ relaxation time was calculated using a single exponential fitting of the echo train. For T₁ measurements TRUE FISP method was used with the following parameters: slice thickness 2 mm, FOV 3×3 cm, 1 average, matrix size 128×128, echo time (T_{E})=1.5 ms, T_{R}=1 s.

### Example 4

### Cytotoxicity

a) Cell Culture

Three human cell types: human umbilical vein endothelial cells (HUVEC, ATCC, Manassas, Va., USA), KB oral carcinoma cells (ATCC) and U343 neuronal glioblastoma cells (supplied by Dr. Ki-Young Lee, Cumming School of Medicine, University of Calgary) were cultured in 12-well plates coated with a thin layer of gelatin (BD Difco, Mississauga, ON, Canada). Endothelial growth medium (EGM-2, Lonza, Hopkinton, Mass., USA), minimum essential medium (MEM, Gibco) and Dulbecco's Modified Eagle Media (DMEM, Gibco, Life Technologies™, Grand Island, N.Y., USA), each containing 10% fetal bovine serum (FBS, PAA Laboratories, Etobicoke, Ontario, Canada) and 100 units/mL PEN/STREP (Gibco), were used to grow the HUVEC, KB and U343 cells respectively. The cells were grown to around 85% confluence prior to exposure with the nanoparticles.

b) Cytotoxicity Testing

Nanoparticle suspensions were prepared for each cell line in their respective media at concentrations ranging from 2.5 to 250 μg/mL. Samples of each cell line were also exposed to growth medium as a negative control and growth medium containing 1.0 μg/mL. InSolution™ Staurosporine (Merck Millipore, Etobicoke, Ontario, Canada) as a positive control. All cell samples were incubated at 37° C. for a total exposure time of 24 hours. Following exposure, all samples were rinsed with Dulbecco's Phosphate-Buffered Saline (DPBS, Fischer Scientific, Ottawa, ON, Canada). Cells were stained with a solution of 10 μM Hoechst 33258 nuclear stain (Molecular Probes™, Life Technologies™, Burlington, ON, Canada) and 10 μM propidium iodide (PI) cell-impermeant stain (Molecular Probes™). Following a 10 minute exposure time to the stain solution, the cells were once again rinsed with DPBS to remove excess stain.

The cells were imaged using a laser scanning confocal microscope (Olympus FluoView™ FV1000, Markham, Ontario, Canada) with a 10× magnification, 0.3 numerical aperture, water immersion objective with 2 digital zoom. Cell viability was determined by comparing the number of PI positive cells to the total number of cells as follows:

$$\% cell viaibility = \frac{{n}_{sample}^{ttl} - {n}_{sample}^{PI}}{{n}_{negative control}^{ttl} - {n}_{negative control}^{PI}} \cdot 100 \% \qquad ( III )$$

Each experimental condition was performed in triplicate. All results are expressed as mean plus standard deviation. One-way analyses of variance (ANOVA) with post-hoc Tukey tests were performed to determine statistical significance of each condition versus the negative control.

It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

All publications mentioned herein are incorporated herein by reference (where permitted) to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
M. Ahmet, C. S. Frampton and J. Silver, J. Chem. Soc., Dalton Trans., 1988, 1159-1163.
J. A. Bau, P. Li, A. J. Marenco, S. Trudel, B. C. Olsen, E. J. Luber and J. M. Buriak, Chem. Mater., 2014, doi:10.1021/cm501881a.
B. Blasiak, T. Foniok, C. Deng and B. Tomanek, J. Neurosc. Meth., 2012, 240, 118-123.
B. Blasiak, F. C. J. M. van Veggel and B. Tomanck, Journal of Nanomaterials, 2013, 2013, 148578.
R. M. Cornell and U. Schwertmann, The Iron Oxides: Structure, Properties, Reactions, Occurences and Uses, Wiley-VCH Verlag GmbH & Co. KGaA, 2nd edn, 2004.
G. K. Das, N. J. J. Johnson, J. Cramen, B. Blasiak, P. Latta, B. Tomanek and F. C. J. M. van Veggel, J. Phys. Chem. Lett., 2012, 3, 524-529.
C. Dong, A. Korinek, B. Blasiak, B. Tomanek and F. C. J. M. van Veggel, Chem. Mater., 2012, 24, 1297-1305.
A. Gupta and S. Wells, IEEE Trans. NanoBioscience, 2004, 3, 66-73.
C. Hanley, J. Layne, A. Punnoose, K. M. Reddy, I. Coombs, A. Coombs, K. Feris and D. Wingett, Nanotechnology, 2008, 19, 295103.
X. Hu, S. Cook, P. Wang, H. min Hwang, X. Liu and Q. L. Williams, Sci. Total Environ., 2010, 408, 1812-1817.
R. M. Cornell and U. Schertwann, 2003, The Iron Oxides: Structure, Properties, Reactions, Occurrences and Uses, Wiley-VCH, Weinheim.
E. Jan, S. J. Byrne, M. Cuddihy, A. M. Davies, Y. Volkov, Y. K. Gunko and N. A. Kotov, ACS Nano, 2008, 2, 928-938.
Y.-w. Jin et al., J. Am. Chem. Soc., 2005, 127, 5732-5733.
Y.-w. Jun, J.-w. Seo and J. Cheon, Accounts of Chemical Research, 2008, 41, 179-189.
C. W. Jung and P. Jacobs, Magn. Reson. Imaging, 1995, 13, 661-674.
W. Lin, Y. wern Huang, X.-D. Zhou and Y. Ma, Toxicol. Appl. Pharmacol., 2006, 217, 252-259.
D. G. Maxton, R. P. H. Thompson and R. C. Hider, Br. J. Nutr., 1994, 71, 203.
H. H. McNeill, V. G. Yuen, H. R. Hoveyda and C. Orvig, J. Med. Chem., 1992, 35, 1489.
N. Lee and T. Hyeon, Chem. Soc. Rev., 2012, 41, 2575-2589.
N. Lee et al., Proc. Natl. Acad. Sci. U.S.A., 2011, 108, 2662-2667.
N. Lewinski, V. Colvin and R. Drezek, Small, 2008, 4, 26-49.
H. B. Na, I. C. Song and T. Hyeon, Adv. Mater., 2009, 21, 2133-2148.
B. S. Parajón-Costa and E. J. Baran, Spectrochim. Acta, Part A, 2013, 113, 337-339.
R. Qiao, C. Yang and M. Gao, J. Mater. Chem., 2009, 19, 6274-6293.
J. Qin, S. Laurent, Y. Jo, A. Roch, M. Mikhaylova, Z. Bhujwalla, R. Muller and M. Muhammed, Adv. Mater., 2007, 19, 1874-1878.
E. Terreno, D. D. Castelli, A. Viale and S. Aime, Chem. Rev., 2010, 110, 3019-3042.
L. C. J. Thomassen, A. Aerts, V. Rabolli, D. Lison, L. Gonzalez, M. Kirsch-Volders, D. Napierska, P. H. Hoet, C. E. A. Kirschhock and J. A. Martens, Langmuir, 2010, 26, 328-335.
K. H. Thompson, C. A. Barta and C. Orvig, Chem, Soc. Rev., 2006, 35, 545-556.
S. Tong, S. Hou, Z. Zheng, J. Zhou and G. Bao, Nano Lett., 2010, 10, 4607-4613.
Y.-X. J. Wang, S. M. Hussain and G. P. Krestin, Eur. Radiol., 2001, 11, 2319-2331.
V. Yathindranath, L. Rebbouh, D. F. Moore, D. W. Miller, J. van Lierop and T. Hegmann, Adv. Funct. Mater., 2011, 21, 1457-1464.
V. Yathindranath, Z. Sun, M. Worden, L. J. Donald, J. A. Thliveris, D. W. Miller and T. Hegmann, Langmuir, 2013, 29, 10850-10858.
Z. Zhao, Z. Zhou, J. Bao, Z. Wang, J. Hu, X. Chi, K. Ni, R. Wang, X. Chen, Z. Chen and J. Gao, Nature Comms., 2013, 4, 2266.

What is claimed is:

1. A nanoparticle comprising a magnetite (Fe₃O₄) core, a non-magnetic layer surrounding the magnetite core, and a maltol coating formed on the non-magnetic layer, wherein the maltol coating comprises a maltol shell surrounding the non-magnetic layer and the magnetite core.

2. The nanoparticle of claim 1, wherein the non-magnetic layer comprises magnetically disordered magnetite (Fe₃O₄).

3. The nanoparticle of claim 2, wherein the non-magnetic layer has a thickness of about 3.5 nm.

4. The nanoparticle of claim 2, wherein the maltol shell comprises at least one maltol moiety bound to the non-magnetic layer.

5. The nanoparticle of claim 4, wherein the maltol moiety is chelated to the non-magnetic layer.

6. The nanoparticle of claim 4, wherein the nanoparticle is superparamagnetic.

7. The nanoparticle of claim 6, wherein the nanoparticle has r₁ relaxation ranging between from about 2.09 to about 2.13 mM⁻¹ s⁻¹.

8. The nanoparticle of claim 6, wherein the nanoparticle has r₂ relaxation ranging between about 176 to about 206 mM⁻¹ s⁻¹.

9. The nanoparticle of claim 6, wherein the nanoparticle exhibits r₂/r₁ of about 90.5 at 9.4 T.

10. The nanoparticle of claim 6, wherein the nanoparticle has r₁ relaxation ranging between from about 0.98 to about 1.08 mM⁻¹ s⁻¹.

11. The nanoparticle of claim 6, wherein the nanoparticle has r₂ relaxation ranging between about 114 to about 138 mM's⁻¹.

12. The nanoparticle of claim 6, wherein the nanoparticle exhibits r₂/r₁ of about 122.0 at 3.0 T.

13. The nanoparticle of claim 6, wherein the nanoparticles are water soluble due to the maltol coating.

14. The nanoparticle of claim 13, wherein the nanoparticle is spherical.

15. The nanoparticle of claim 14, wherein the nanoparticle has a diameter ranging between about 5 nm to about 15 nm.

16. The nanoparticle of claim 15, wherein the nanoparticle is non-toxic and clinically applicable.

17. A pharmaceutical composition comprising the nanoparticle of claim 1 in combination with one or more pharmaceutically acceptable carriers.

18. A magnetic resonance imaging contrast agent for detecting an in vivo site by magnetic resonance imaging, comprising the nanoparticle of claim 1.

19. A method of in vivo imaging of a site within a subject comprising administering to the subject a nanoparticle, or a pharmaceutical composition comprising the nanoparticle in combination with one or more pharmaceutically acceptable carriers; and imaging the site of the subject.

20. The method of claim 19, wherein the imaging comprises magnetic resonance imaging.

21. The method of claim 19, wherein the in vivo site is selected from the heart, liver, biliary tract, kidney, spleen, bowel, pancreas, adrenal gland, bladder, uterus, ovary, prostate gland, blood vessel, or lymph node.

22. The method of claim 19, wherein the nanoparticle, or the pharmaceutical composition comprising the nanoparticle is used for diagnosis and monitoring treatment of a tumor, a liver disease, an abnormality of the bile ducts or pancreas, inflammatory bowel disease, a heart problem, a malformation or inflammation of a blood vessel, or development of a fetus.
